(19) United States
(12) Patent Application Publication
(10) Pub. No.: US 2019/0191732 A1
(43) Pub. Date: Jun. 27, 2019

(54) Ketogenic Dietary Consumable

(71) Applicant: Bill McIntosh, Huntington Beach, CA (US)

(72) Inventor: Bill McIntosh, Huntington Beach, CA (US)

(21) Appl. No.: 16/230,950

(22) Filed: Dec. 21, 2018

Related U.S. Application Data
(60) Provisional application No. 62/608,941, filed on Dec. 21, 2017.

(51) Int. Cl. A23D 9/04; A23F 5/46; A23L 33/12; A61K 36/42; A61K 36/889; A61K 36/54

(52) U.S. Cl. CPC A23D 9/04; A23F 5/46; A23L 33/12; A61K 36/42; A61K 36/889; A23V 2250/1944; A23V 2002/00; A23V 2200/332; A23V 2250/2108; A23V 2250/1942; A61K 36/54

(57) ABSTRACT

A ketogenic dietary consumable provides optimal fats to a consumer who is on the ketogenic diet. The ketogenic dietary consumable utilizes both medium-chain triglycerides (MCTs) and long-chain triglycerides (LCTs) in order to provide necessary calories and avoid stomach discomfort and gastrointestinal distress associated with less triglyceride-diverse dietary supplements. The ketogenic dietary consumable includes a quantity of fractionated coconut oil, a quantity of avocado oil, and a quantity of macadamia nut oil. The quantity of fractionated coconut oil provides the ketogenic dietary consumable with valuable MCTs, which are easily digestible. The quantity of avocado oil and the quantity of macadamia nut oil supply the ketogenic dietary consumable with a strong source of LCTs, thus preventing the rapid digestion of the MCTs from the quantity of fractionated coconut oil that results in stomach discomfort.

The current application claims a priority to the U.S. Provisional Patent application Ser. No. 62/608,941 filed on Dec. 21, 2017.

### FIELD OF THE INVENTION

The present invention relates generally to a dietary supplement. More specifically, the ketogenic dietary consumable relates to a mixture of various fatty acids that prevents stomach discomfort common in people who consume common oil supplements. The present invention is particularly advantageous for people who take part in the popular ketogenic diet, which requires high fat input.

### BACKGROUND OF THE INVENTION

For people attempting to lose weight and improve physical health, the most common and recommended solutions are diet and exercise. Among diets that are popular for reducing weight and achieving higher physical and mental performance is the ketogenic diet. By removing carbs and increasing intake of fats, the user can induce a state in which their bodies convert fats into energy. The body is capable of producing its own carbs without ingesting them externally, so not eating extra carbs does not have harmful effects in most healthy people.

Achieving ketosis, however, is a difficult feat. The body often takes several days to adjust to the new input of high fats without any carbs. This often leaves the user feeling fatigued or otherwise mentally exhausted, during the adjustment period. To this end, a variety of ketogenic formulations and supplements have been developed. These formulations tend to be focused around providing the user with medium chain triglycerides, a set of fats which are commonly found in some oils derived from coconuts. By receiving concentrated doses of these oils, the user can ease the transition from carbs considerably, by providing ample material from which the body can create energy. However, formulations that depend on strictly medium chain triglyceride oils, also known as MCT oils, may interfere with a user's digestion, causing various intestinal issues and stomach discomfort. This is primarily because the body is not equipped to efficiently consume large amounts of concentrated MCT oil. What is needed is an MCT oil supplement that avoids the issue of causing stomach upset and diarrhea and simultaneously addresses the need for a formulation that enables users to overcome the difficulty of achieving ketosis.

The present invention addresses these issues. The present invention adds oleic acid, palmitoleic acid, alpha-linolenic acid, and linolenic acid to the established MCT oil. These fatty acids are derived from avocados and from macadamia nuts. By blending a specific combination of fatty acids with MCTs, the user enables easier digestion of the fatty oils that will become the user's primary source of fuel and thus eases stomach upset. This enables the user to ingest higher amounts of MCT oils, which prevents the user from becoming hungry and facilitates the body's transition to consuming fats instead of carbs. The avocado and macadamia nut-based oils help to normalize glucose and insulin levels, which can be used to help people with diabetes manage their condition. These oils also decrease cholesterol, which is good for countering the cholesterol-raising effect of lauric acid, which is also commonly found in MCT oil formulas.

### BRIEF DESCRIPTION OF THE DRAWINGS

FIG. 1 is a block diagram depicting the ingredients of the present invention.

FIG. 2 is a pie chart depicting the ratio of the ingredients in an exemplary embodiment of the present invention.

FIG. 3 is a block diagram depicting the fatty acid constituents of the quantity of fractionated coconut oil.

FIG. 4 is a block diagram depicting the fatty acid constituents of the quantity of avocado oil and the quantity of macadamia nut oil.

FIG. 5 is a block diagram depicting the fatty acid constituents of the quantity of coffee bean oil and the quantity of pumpkin seed oil.

### DETAILED DESCRIPTION OF THE INVENTION

All illustrations of the drawings are for the purpose of describing selected versions of the present invention and are not intended to limit the scope of the present invention.

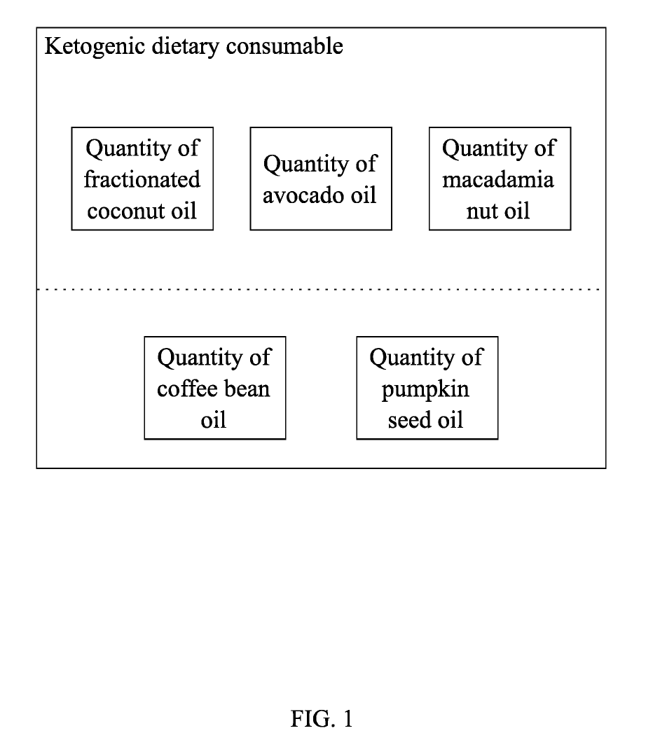

The present invention is a ketogenic dietary consumable for providing fats to a consumer on the ketogenic diet. The present invention utilizes both medium-chain triglycerides (MCTs) and long-chain triglycerides (LCTs) in order to provide necessary calories and avoid stomach discomfort and gastrointestinal distress associated with less triglyceride-diverse dietary supplements. The present invention comprises a quantity of fractionated coconut oil, a quantity of avocado oil, and a quantity of macadamia nut oil, as seen in FIG. 1. The quantity of fractionated coconut oil provides the present invention with valuable MCTs, which are easily digestible. The quantity of avocado oil and the quantity of macadamia nut oil supply the present invention with a strong source of LCTs, thus preventing the rapid digestion of the MCTs from the quantity of fractionated coconut oil that results in stomach discomfort. The quantity of fractionated coconut oil, the quantity of avocado oil, and the quantity of macadamia nut oil includes, but is not limited to, oleic acid, palmitoleic acid, linolenic acid, alpha-linolenic acid, caprylic acid, capric acid, and lauric acid, which are each useful and digestible by the user. The quantity of fractionated coconut oil, the quantity of avocado oil, and the quantity of macadamia nut oil are mixed into a dietary mixture. This arrangement allows for the presence of MCTs and LCTs in the dietary mixture. In an exemplary embodiment, the quantity of fractionated avocado oil is blended with the quantity of avocado oil for one hour and left alone for 24 hours. Then, the quantity of macadamia nut oil is blended with the combined quantity of fractionated avocado oil and quantity of avocado oil for one hour. The dietary mixture is the end result of leaving this mixture alone again for 24 hours.

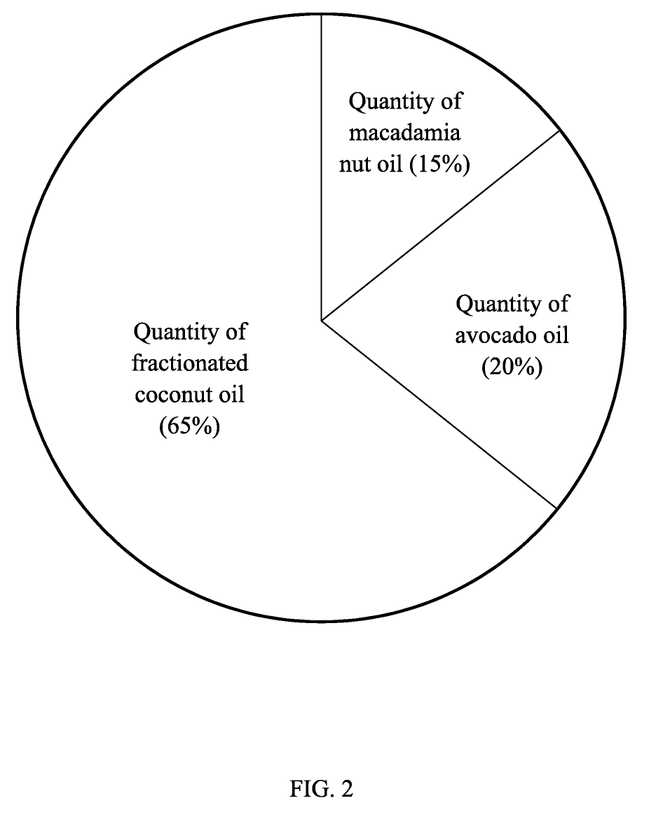

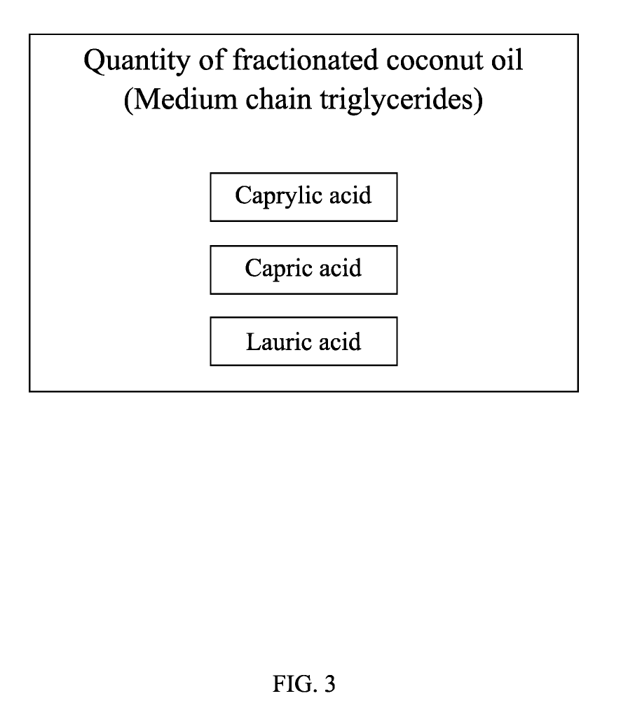

In order to provide an accurate combination of fats to promote and sustain ketosis, the present invention requires the substantial presence of MCTs. To this end, the quantity of fractionated coconut oil ranges between 54 percent volume (vol %) and 71 vol % of the dietary mixture, as seen in FIG. 2. The quantity of fractionated coconut oil contains caprylic acid, capric acid, and lauric acid, as seen in FIG. 3. Within the specified ranges, the quantity of fractionated coconut oil provides an adequate amount of these MCTs without overwhelming the user's digestive system.

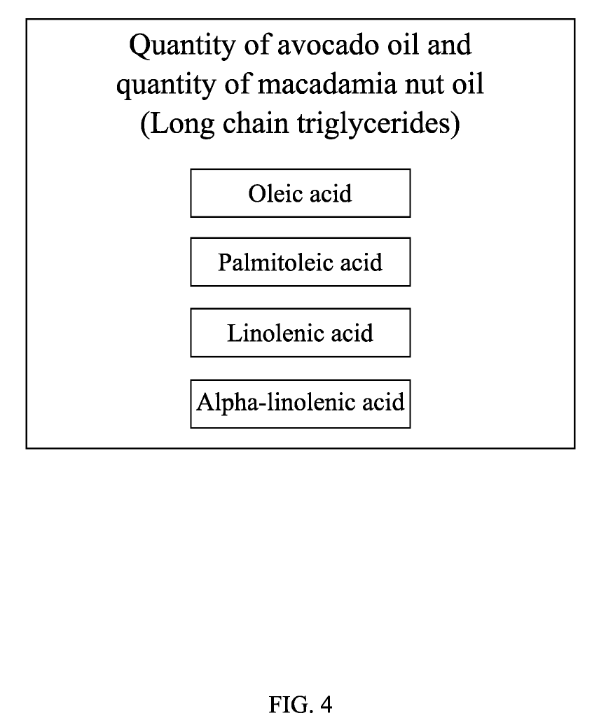

To balance the MCTs provided by the quantity of fractionated coconut oil and provide an accurate combination of fats to promote and sustain ketosis, the present invention requires the presence of LCTs. Therefore, the quantity of avocado oil ranges between 16 vol % and 25 vol % of the dietary mixture. Furthermore, the quantity of macadamia nut oil ranges between 12 vol % and 19 vol % of the dietary mixture, as seen in FIG. 2. The quantity of avocado oil and the quantity of macadamia nut oil provide ample oleic acid, palmitoleic acid, linolenic acid, and alpha-linolenic acid, as seen in FIG. 4. These acids combine to slow digestion of the MCTs found in the quantity of fractionated coconut oil. The oleic acid, palmitoleic acid, linolenic acid, and alpha-linolenic acid further combine to aid in conversion of the caprylic acid, the capric acid, and the lauric acid into energy.

In some embodiments of the present invention, the amount of MCTs from the quantity of fractionated coconut oil may outweigh the amount of LCTs from the quantity of avocado oil and the quantity of macadamia nut oil. In such a circumstance, it may be advantageous to supplement the dietary mixture with supplemental LCTs. To this end, the present invention further comprises a quantity of coffee bean oil, as seen in FIG. 1. The quantity of coffee beam oil is a fatty substance that provides digestion-slowing LCTs. The quantity of coffee bean oil is mixed into the dietary mixture. This arrangement ensures that the LCTs within the coffee bean oil are available to mix and interact with the MCTs from the quantity of fractionated coconut oil. Furthermore, the quantity of coffee bean oil ranges between 5 vol % and 10 vol % of the dietary mixture. In this way, the dietary mixture retains an appropriate concentration of MCTs relative to the amount of LCTs.

In some other embodiments of the present invention, the amount of MCTs from the quantity of fractionated coconut oil may outweigh the amount of LCTs from the quantity of avocado oil and the quantity of macadamia nut oil. In such a circumstance, it may be advantageous to supplement the dietary mixture with supplemental LCTs. To this end, the present invention comprises a quantity of pumpkin seed oil, as seen in FIG. 1. The quantity of pumpkin seed oil is another fatty substance that provides digestion-slowing LCTs. The quantity of pumpkin seed oil is mixed into the dietary mixture. This arrangement ensures that the LCTs within the pumpkin seed oil are available to mix and interact with the MCTs from the quantity of fractionated coconut oil. Furthermore, the quantity of pumpkin seed oil ranges between 5 vol % and 10 vol % of the dietary mixture. In this way, the dietary mixture retains an appropriate concentration of MCTs relative to the amount of LCTs.

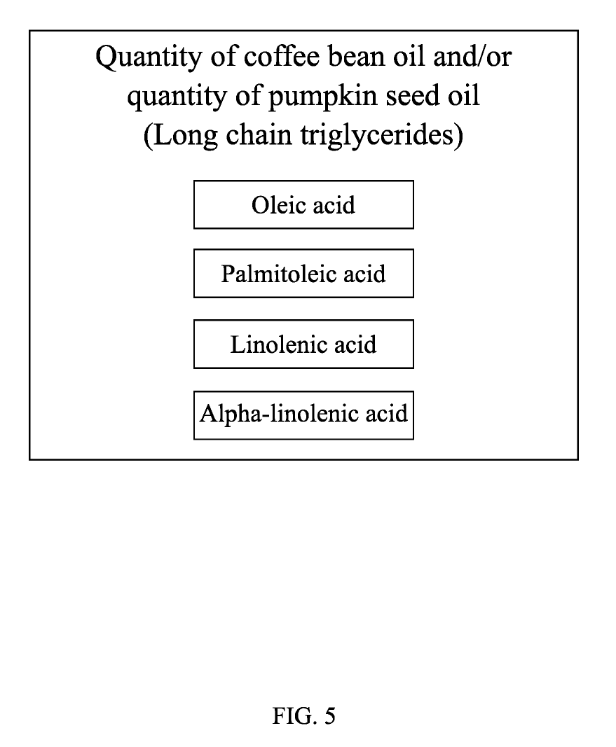

The different textures and flavors of the coffee bean oil and the pumpkin seed oil may make one more desirable than the other; however, in order to dilute the amount of MCTs within the final dietary mixture while including both flavors and textures, some other embodiments of the present invention use a combination of both the coffee bean oil and the pumpkin seed oil. To this end, the present invention further comprises a quantity of coffee bean oil and a quantity of pumpkin seed oil. The quantity of coffee bean oil and the quantity of pumpkin seed oil both provide LCTs, including, but not limited to, oleic acid, palmitoleic acid, linolenic acid, and alpha-linolenic acid, as seen in FIG. 5, which aid in preventing uncomfortably quick digestion of MCTs. The quantity of coffee bean oil and the quantity of pumpkin seed oil are both mixed into the dietary mixture. This allows the quantity of coffee bean oil and the quantity of pumpkin seed oil to affect the quantity of fractionated coconut oil, the quantity of avocado oil, and the quantity of macadamia nut oil. In the preferred embodiment, a combination of the quantity of coffee bean oil and the quantity of pumpkin seed oil ranging between 5 vol % and 10 vol % of the dietary mixture is utilized. This relatively low percentage ensures that the ratio of MCTs to LCTs remains within an optimal range.

The method for manufacturing the ketogenic formulation comprises an ingredient assembly step, a first combination step, a first blending step, a second combination step, a second blending step, and a third blending step. In the ingredient assembly step, the user collects the ingredients necessary for creating the ketogenic formulation. In an exemplary embodiment, these ingredients comprise a quantity of fractionated coconut oil, a quantity of avocado oil, and a quantity of macadamia nut oil. In alternative embodiments, the ingredients to be assembled further comprise a quantity of coffee bean oil and/or a quantity of pumpkin seed oil. The user gathers 65 vol % fractionated coconut oil, 20 vol % avocado oil, and 15 vol % macadamia nut oil. In the alternative embodiment that includes coffee bean oil and/or pumpkin seed oil, the user includes approximately 5 vol % relative to the combined product of the quantity of fractionated coconut oil, the quantity of avocado oil, and the quantity of macadamia nut oil. The user then performs the first combination step. The user combines the quantity of fractionated coconut oil with the quantity of avocado oil. Next, the user begins the first blending step. The user blends the quantity of fractionated coconut oil with the quantity of avocado oil for approximately one hour. The user then allows the mixture to sit for approximately 24 hours. The user then begins the second combination step. In the preferred embodiment, the user adds the quantity of macadamia nut oil to the quantity of fractionated coconut oil and quantity of avocado oil mixture. In an alternative embodiment, the user adds the quantity of macadamia nut oil with either the quantity of coffee bean oil, the quantity of pumpkin seed oil, or both, to the quantity of fractionated coconut oil and quantity of avocado oil mixture. The user then begins the second blending step. The user blends the mixture for approximately one hour. The user then leaves the mixture to rest for approximately 24 hours. The final step is the third blending step. The user blends the mixture for approximately one hour before the mixture is prepared for subsequent packaging and marketing.

In the preferred usage of the present invention, the user acquires the present invention. The user prepares to brace the user's body by disposing of all high carb foods, and replacing them with high fat low carb foods, such as fish, meat, and certain vegetables. The user then ingests the present invention. The user may add the present invention to cooking recipes, to drinks such as smoothies, or even raw as oil for salads or in other foods. Approximately one tablespoon is generally considered a serving for this and most MCT-based oils. By adding the present invention to a variety of foods during the day, the user can provide the user's body with ample energy and fat to convert into energy for the day and can also control the user's insulin and blood sugar levels.

Although the invention has been explained in relation to its preferred embodiment, it is to be understood that many other possible modifications and variations can be made without departing from the spirit and scope of the invention as hereinafter claimed.

What is claimed is:

1. A ketogenic dietary consumable comprises:
a quantity of fractionated coconut oil;
a quantity of avocado oil;
a quantity of macadamia nut oil; and
the quantity of fractionated coconut oil, the quantity of avocado oil, and the quantity of macadamia nut oil being mixed into a dietary mixture.

2. The ketogenic dietary consumable as claimed in claim 1 comprises:
the quantity of fractionated coconut oil ranging between 54 percent volume (vol %) and 71 vol % of the dietary mixture.

3. The ketogenic dietary consumable as claimed in claim 1 comprises:
the quantity of avocado oil ranging between 16 vol % and 25 vol % of the dietary mixture.

4. The ketogenic dietary consumable as claimed in claim 1 comprises:
the quantity of macadamia nut oil ranging between 12 vol % and 19 vol % of the dietary mixture.

5. The ketogenic dietary consumable as claimed in claim 1 comprises:
a quantity of coffee bean oil; and
the quantity of coffee bean oil being mixed into the dietary mixture.

6. The ketogenic dietary consumable as claimed in claim 5 comprises:
the quantity of coffee bean oil ranging between 5 vol % and 10 vol % of the dietary mixture.

7. The ketogenic dietary consumable as claimed in claim 1 comprises:
a quantity of pumpkin seed oil; and
the quantity of pumpkin seed oil being mixed into the dietary mixture.

8. The ketogenic dietary consumable as claimed in claim 7 comprises:
the quantity of pumpkin seed oil ranging between 5 vol % and 10 vol % of the dietary mixture.

9. The ketogenic dietary consumable as claimed in claim 1 comprises:
a quantity of coffee bean oil;
a quantity of pumpkin seed oil; and
the quantity of coffee bean oil and the quantity of pumpkin seed oil being mixed into the dietary mixture.

10. The ketogenic dietary consumable as claimed in claim 9 comprises:
a combination of the quantity of coffee bean oil and the quantity of pumpkin seed oil ranging between 5 vol % and 10 vol % of the dietary mixture.

11. A ketogenic dietary consumable comprises:
a quantity of fractionated coconut oil;
a quantity of avocado oil;
a quantity of macadamia nut oil;
the quantity of fractionated coconut oil, the quantity of avocado oil, and the quantity of macadamia nut oil being mixed into a dietary mixture; and
the quantity of fractionated coconut oil ranging between 54 percent volume (vol %) and 71 vol % of the dietary mixture.

12. The ketogenic dietary consumable as claimed in claim 11 comprises:
the quantity of avocado oil ranging between 16 vol % and 25 vol % of the dietary mixture.

13. The ketogenic dietary consumable as claimed in claim 11 comprises:
the quantity of macadamia nut oil ranging between 12 vol % and 19 vol % of the dietary mixture.

14. The ketogenic dietary consumable as claimed in claim 11 comprises:
a quantity of coffee bean oil;
the quantity of coffee bean oil being mixed into the dietary mixture; and
the quantity of coffee bean oil ranging between 5 vol % and 10 vol % of the dietary mixture.

15. The ketogenic dietary consumable as claimed in claim 11 comprises:
a quantity of pumpkin seed oil;
the quantity of pumpkin seed oil being mixed into the dietary mixture; and
the quantity of pumpkin seed oil ranging between 5 vol % and 10 vol % of the dietary mixture.

16. The ketogenic dietary consumable as claimed in claim 11 comprises:
a quantity of coffee bean oil;
a quantity of pumpkin seed oil;
the quantity of coffee bean oil and the quantity of pumpkin seed oil being mixed into the dietary mixture; and
a combination of the quantity of coffee bean oil and the quantity of pumpkin seed oil ranging between 5 vol % and 10 vol % of the dietary mixture.

17. A ketogenic dietary consumable comprises:
a quantity of fractionated coconut oil;
a quantity of avocado oil;
a quantity of macadamia nut oil;
the quantity of fractionated coconut oil, the quantity of avocado oil, and the quantity of macadamia nut oil being mixed into a dietary mixture;
the quantity of fractionated coconut oil ranging between 54 percent volume (vol %) and 71 vol % of the dietary mixture;
the quantity of avocado oil ranging between 16 vol % and 25 vol % of the dietary mixture; and
the quantity of macadamia nut oil ranging between 12 vol % and 19 vol % of the dietary mixture.

18. The ketogenic dietary consumable as claimed in claim 17 comprises:
a quantity of coffee bean oil;
the quantity of coffee bean oil being mixed into the dietary mixture; and
the quantity of coffee bean oil ranging between 5 vol % and 10 vol % of the dietary mixture.

19. The ketogenic dietary consumable as claimed in claim 17 comprises:
a quantity of pumpkin seed oil;
the quantity of pumpkin seed oil being mixed into the dietary mixture; and
the quantity of pumpkin seed oil ranging between 5 vol % and 10 vol % of the dietary mixture.

20. The ketogenic dietary consumable as claimed in claim 17 comprises:
a quantity of coffee bean oil;
a quantity of pumpkin seed oil;
the quantity of coffee bean oil and the quantity of pumpkin seed oil being mixed into the dietary mixture; and
a combination of the quantity of coffee bean oil and the quantity of pumpkin seed oil ranging between 5 vol % and 10 vol % of the dietary mixture.
